(19) United States
(12) Patent Application Publication
(10) Pub. No.: US 2025/0345488 A1
(43) Pub. Date: Nov. 13, 2025

(54) CROSSLINKED HYDROGELS WITH ENHANCED RADIOPACITY FOR MEDICAL APPLICATIONS

(71) Applicant: Boston Scientific Scimed, Inc., Maple Grove, MN (US)

(72) Inventor: Yen-Hao Hsu, Shrewsbury, MA (US)

(73) Assignee: Boston Scientific Scimed, Inc., Maple Grove, MN (US)

(21) Appl. No.: 19/205,442

(22) Filed: May 12, 2025

Related U.S. Application Data
(60) Provisional application No. 63/646,320, filed on May 13, 2024.

(51) Int. Cl. A61L 27/52; A61L 27/58; C08J 3/075; C08J 3/24

(52) U.S. Cl. CPC A61L 27/52; A61L 27/58; C08J 3/075; C08J 3/242; A61L 2400/06; C08J 2371/08; C08J 2427/10

(57) ABSTRACT

In some aspects, the present disclosure provides a radiopaque, reactive polymer comprising one or more hydrophilic polymer regions, the one or more hydrophilic polymer regions having first and second ends and comprising at least one hydrophilic polymer segment, a plurality of iodinated amino-acid residues, wherein at least one of the plurality of iodinated amino-acid residues is linked to each of the hydrophilic polymer regions, and a plurality of reactive moieties, wherein at least one of the plurality of reactive moieties is linked to each of the plurality of iodinated amino-acid residues and wherein the reactive moieties each comprise an electrophilic group. In other aspects the present disclosure pertains to systems that comprise such a radiopaque, reactive polymer and a compound having a plurality of nucleophilic moieties that reacts with the radiopaque, reactive polymer to form a crosslinked reaction product, as well as methods of treatment using the same.

### CROSS-REFERENCE TO RELATED APPLICATIONS

This application claims the benefit of U.S. Provisional Patent Application Ser. No. 63/646,320 filed on May 13, 2024, the disclosure of which is incorporated herein by reference.

### FIELD

The present disclosure relates to radiopaque hydrogels and to crosslinkable systems for forming radiopaque hydrogels, among other aspects. The radiopaque hydrogels and crosslinkable systems for forming the same are useful, for example, in various medical applications

### BACKGROUND

Bioresorbable hydrogels with rapid crosslinking reaction rate in vivo, known by the trade name of SpaceOAR®, have become a prominent biomaterial and obtained clinical success in creating the space between prostate and rectum, tremendously improving patient safety during the cancer therapies. SpaceOAR® is based on a multi-arm polyethylene glycol (PEG) polymer with a polyol core functionalized with succinimidyl glutarate (SG) as reactive end groups which further react with trilysine to form crosslinks. A further improvement based on this application is that some of 8-Arm PEG branches are functionalized with 2,3,5-triiiodobenzamide (TIB) groups, replacing part of the SG groups, in order to provide intrinsic radiopacity to the hydrogels themselves for CT-visibility. This hydrogel, known by the trade name of SpaceOAR Vue®, is the next generation of SpaceOAR® for prostate medical applications. The hydrogels break down in-vivo over the course of ca. 6-9 months. The breakdown occurs primarily through the hydrolysis of the ester linkages on the glutarate groups.

While the above approach is effectual, the entire functionalization process is complex, involving multiple steps, typically five steps, from commercially available hydroxyl-terminated 8-arm PEG to its functionalized form with two different end groups (TIB and SG groups) and resulting in a significant increase of the product cost. Moreover, in order to functionalize TIB on 8-arm PEG, one SG group is sacrificed, reducing the number of crosslinks per 8-arm PEG molecule.

Alternative strategies for forming iodine-labelled crosslinked hydrogels that provide enhanced and tunable radiopacity while maintaining crosslink density per polymer molecule are desired.

### SUMMARY

In some aspects, the present disclosure pertains to radiopaque reactive polymers that comprise one or more hydrophilic polymer regions, the one or more hydrophilic polymer regions having first and second ends and comprising (a) at least one hydrophilic polymer segment, (b) a plurality of iodinated amino-acid residues, where at least one of the plurality of iodinated amino-acid residues is linked to each of the hydrophilic polymer regions, and (c) a plurality of reactive moieties, where at least one of the plurality of reactive moieties is linked to each of the plurality of iodinated amino-acid residues.

In some embodiments, the reactive polymer is a linear polymer in which the one or more hydrophilic polymer regions correspond to a central linear hydrophilic polymer region and in which one of the plurality of iodinated amino-acid residues is linked to each of the first and second ends of the central linear hydrophilic polymer region.

In some embodiments, the reactive polymer is a multi-arm polymer comprising a core region and a plurality of polymer arms, wherein the plurality of polymer arms each comprise one of the one or more hydrophilic polymer regions, wherein the first end is linked to the core region, and wherein the second end is linked to one of the plurality of iodinated amino-acid residues.

In some embodiments, which can be used in conjunction with the above aspects and embodiments, each of the plurality of iodinated amino-acid residues is linked to one of the one or more hydrophilic polymer regions through an ester group and each of the plurality of reactive moieties is linked to one of the plurality of iodinated amino-acid residues though an amide group.

In some embodiments, which can be used in conjunction with the above aspects and embodiments, each of the plurality of iodinated amino-acid residues comprises an iodinated aromatic group.

In some embodiments, which can be used in conjunction with the above aspects and embodiments, each of the plurality of iodinated amino-acid residues comprises a hydroxy-iodo-aromatic group. For example, the hydroxy-iodo-phenyl group may be selected from a mono-hydroxy-mono-iodo-phenyl group, a mono-hydroxy-di-iodo-phenyl group, a mono-hydroxy-tri-iodo-phenyl group, a mono-hydroxy-tetra-iodo-phenyl group, a di-hydroxy-mono-iodo-phenyl group, a di-hydroxy-di-iodo-phenyl group, a di-hydroxy-tri-iodo-phenyl group, a di-hydroxy-tetra-iodo-phenyl group, a tri-hydroxy-mono-iodo-phenyl group, a tri-hydroxy-di-iodo-phenyl group, tri-hydroxy-tri-iodo-phenyl group, and a tri-hydroxy-tetra-iodo-phenyl group, among others.

In some embodiments, which can be used in conjunction with the above aspects and embodiments, the iodinated amino-acid residues are selected from mono-iodophenylalanine, di-iodophenylalanine, tri-iodophenylalanine, mono-iodoiodotyrosine, di-iodoiodotyrosine, tri-iodoiodotyrosine, mono-iodothyronine, di-iodothyronine, tri-iodothyronine, and tetra-iodothyronine residues.

In some embodiments, which can be used in conjunction with the above aspects and embodiments, the reactive moiety comprises an electrophilic group.

In some embodiments, which can be used in conjunction with the above aspects and embodiments, the reactive moiety comprises a cyclic imide ester group. For example, cyclic imide ester group may be selected from a succinimide ester group, a maleimide ester group, a glutarimide ester group, a diglycolimide ester group, a phthalimide ester group, and a bicyclo[2.2.1]hept-5-ene-2,3-dicarboxylic acid imide ester group, among others.

In some embodiments, which can be used in conjunction with the above aspects and embodiments, the radiopaque, reactive polymer comprises N-(cyclic-imidyl-oxycarbonyl-C₁-C₆-alkyl-carbonyl)-substituted iodinated amino acid residues, N-(cyclic-imidyl-oxycarbonyl-C₂-C₆-alkenyl-carbonyl)-substituted iodinated amino acid residues, or N-(cyclic-imidyl-oxycarbonyl-C₂-C₆-alkyloxyalkyl-carbonyl)-substituted iodinated amino acid residues. For example, the radiopaque, reactive polymer may comprise succinimidyl glutaramide groups, succinimidyl malonamide groups, succinimidyl succinamide groups, succinimidyl adipamide groups, succinimidyl pimelamide groups, or succinimidyl diglycolamide groups, among others.

In some embodiments, which can be used in conjunction with the above aspects and embodiments, the at least one hydrophilic polymer segment comprises one or more monomer residues selected from ethylene oxide, propylene oxide, N-vinyl pyrrolidone and oxazoline monomer residues.

In some embodiments, which can be used in conjunction with the above aspects and embodiments, the at least one hydrophilic polymer segment contains between 40 and 4000 monomer residues.

Other aspects of the present disclosure pertain to systems for forming hydrogel compositions that comprise (a) a compound having a plurality of nucleophilic moieties and (b) a radiopaque, reactive polymer in accordance with any of the above aspects and embodiments.

In some embodiments, the compound having a plurality of nucleophilic moieties is a polyamino compound.

In some embodiments, which can be used in conjunction with the above aspects and embodiments, the systems comprise a first composition that comprises the compound having a plurality of nucleophilic moieties and a second composition that comprises the radiopaque, reactive polymer. In some of these embodiments, the system may further comprise an accelerant composition.

In some embodiments, which can be used in conjunction with the above aspects and embodiments, the systems further comprise a delivery device.

In other aspects, the present disclosure pertains to methods of treatment that comprise administering to a subject a mixture that comprises (a) a radiopaque, reactive polymer in accordance with any of the above aspects and embodiments and (b) a compound having a plurality of nucleophilic moieties, wherein the mixture is administered under conditions such that the compound having a plurality of nucleophilic moieties and the radiopaque, reactive polymer crosslink to form a hydrogel after administration.

Potential benefits associated with the present disclosure include one or more of

the following: radiocontrast is maintained, crosslink density is enhanced, and in vivo persistence is obtained.

The above and other aspects, embodiments, features and benefits of the present disclosure will be readily apparent from the following detailed description.

### BRIEF DESCRIPTION OF THE DRAWINGS

FIG. 1 schematically illustrates a method of forming a radiopaque, reactive polymer, in accordance with an embodiment of the present disclosure.

FIG. 2 schematically illustrates a method of forming a radiopaque crosslinked hydrogel, in accordance with an embodiment of the present disclosure.

FIG. 3 illustrates a delivery device, in accordance with an embodiment of the present disclosure.

FIG. 4 illustrates a delivery device, in accordance with another embodiment of the present disclosure.

### DETAILED DESCRIPTION

The present disclosure pertains to radiopaque, reactive polymers including radiopaque, reactive linear polymers and radiopaque, reactive multi-arm polymers. As discussed below, the radiopaque, reactive polymers have inherent radiopacity without loss of reactive groups.

Radiopaque, reactive polymers in accordance with the present disclosure include polymers that comprise one or more hydrophilic polymer regions, the one or more hydrophilic polymer regions having first and second ends and comprising at least one hydrophilic polymer segment, a plurality of iodinated amino-acid residues linked to one or more hydrophilic polymer regions, and a plurality of reactive moieties, where one of the plurality of reactive moieties is linked to each of the plurality of iodinated amino-acid residues.

Radiopaque, reactive linear polymers in accordance with the present disclosure include polymers that comprise a central linear hydrophilic polymer region comprising a hydrophilic polymer segment, an iodinated amino-acid residue linked to each end of the central linear hydrophilic polymer region, and a reactive moiety that is linked to each iodinated amino-acid residue. In various embodiments, each iodinated amino-acid residue is linked to each end of the central linear hydrophilic polymer region through an ester group, and each reactive moiety is linked to each iodinated amino-acid residue though an amide group.

Radiopaque, reactive multi-arm polymers in accordance with the present disclosure include polymers that comprise a plurality of polymer arms linked to a core region. The polymer arms each comprise a linear hydrophilic polymer region having first and second ends and comprising a hydrophilic polymer segment. The first end of the linear hydrophilic polymer region is linked to the core region and the second end of the linear hydrophilic polymer region is linked to an iodinated amino- acid residue. A reactive moiety is covalently linked to the iodinated amino-acid residue. In various embodiments, each iodinated amino-acid residue is linked to each linear hydrophilic polymer region through an ester group, and each reactive moiety is linked to each iodinated amino-acid residue though an amide group.

Radiopaque, reactive multi-arm polymers include polymers having from 3 to 100 polymer arms, for example ranging anywhere from 3 to 4 to 5 to 6 to 7 to 8 to 9 to 10 to 11 to 12 to 15 to 20 to 25 to 50 to 75 to 100 polymer arms (in other words, having a number of polymer arms ranging between any two of the preceding values).

In some embodiments, the reactive moiety comprises an electrophilic group. Electrophilic groups may be selected, for example, from cyclic imide ester groups, such as succinimide ester groups,

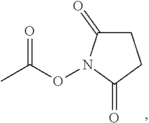

maleimide ester groups, glutarimide ester groups, diglycolimide ester groups, phthalimide ester groups, and bicyclo[2.2.1]hept-5-ene-2,3-dicarboxylic acid imide ester groups,

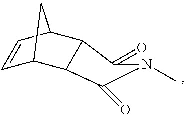

imidazole ester groups, imidazole carboxylate groups and benzotriazole ester groups, among other possibilities.

Hydrophilic polymer segments can be selected from any of a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinyl imidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain from 10 monomer units or less to 1000 monomer units or more, for example, ranging anywhere from 5 to 10 to 20 to 50 to 100 to 200 to 500 to 1000 to 2000 monomer units.

As previously noted, in the case of radiopaque, reactive multi-arm polymers, the polymer arms extend from a core region. In certain of these embodiments, the core region comprises a residue of a polyol comprising two or more hydroxyl groups, which is used to form the polymer arms.

In certain embodiments, the core region comprises a residue of a polyhydroxy compound comprising three or more hydroxyl groups, also referred to herein as a “polyol”, which is used to form the polymer arms. For example, the core region may comprise a residue of a polyol that contains from 3 to 100 hydroxyl groups, for example ranging anywhere from 3 to 4 to 5 to 6 to 7 to 8 to 9 to 10 to 11 to 12 to 15 to 20 to 25 to 50 to 75 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, tetrapentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

Polyols having a biodegradable ester group may also be made from polyols such those described above. For example, a precursor polyol such as those described above may be reacted in a ring-opening reaction with a lactone (e.g., α-acetolactone, β-propiolactone, γ-butyrolactone, δ-valerolactone, ε-caprolactone, etc.) to form a further polyol that comprises a hydroxyl end group that is linked to a residue of precursor polyol through an alkyl group (e.g., a C₁-C₁₀ alkyl group) and a hydrolysable ester group at the site of each of the hydroxyl groups of polyol.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

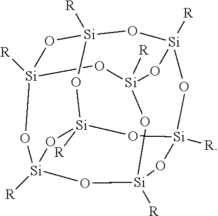

In the present disclosure, the R groups comprise the polymer arms described herein.

Radiopaque, reactive linear polymers in accordance with the present disclosure can be formed from hydroxy-terminated precursor linear polymers. Likewise, radiopaque, reactive multi-arm polymers in accordance with the present disclosure can be formed from hydroxy-terminated precursor multi-arm polymers having arms that comprise one or more hydroxyl end groups.

In some embodiments, a hydroxy-terminated linear hydrophilic polymer or a hydroxy-terminated multi-arm hydrophilic polymer may be reacted with a carboxylic acid group of an iodinated amino acid to form an amino-acid-terminated linear hydrophilic polymer or an amino-acid-terminated multi-arm hydrophilic polymer, in which terminal amino-acid-residues resulting from the iodinated amino acids are linked a remainder of the polymer through a hydrolysable ester group.

Examples of iodinated amino acids include iodinated alpha-amino acids, iodinated beta-amino acids, iodinated gamma-amino acids and iodinated delta-amino acids. It should be noted that although iodine atoms are described, other radiopaque atoms can be employed in place of the iodine atoms, including bromine.

An iodinated amino acid is an amino acid in which the side group contains one or more iodine atoms.

In various embodiments, the side group of the iodinated amino acid comprises one, two, three, or more iodinated aromatic groups. Examples of iodinated aromatic groups include iodine-substituted monocyclic aromatic groups and iodine-substituted multicyclic aromatic groups, such as, for example, iodine-substituted phenyl groups and iodine-substituted naphthyl groups. The aromatic groups may be substituted with one, two, three, four, five, six or more iodine atoms. In various embodiments, the aromatic groups may be further substituted with one or more hydrophilic groups, for example, one, two, three, four, five, six or more hydrophilic groups. The hydrophilic groups may comprise, for example, one or more of the following groups: hydroxyl groups, hydroxyalkyl groups (e.g., hydroxyalkyl groups containing one carbon, two carbons, three carbons, four carbons, etc.), and/or ester groups (e.g., ester groups containing two carbons, three carbons, four carbons, five carbons, six carbons, etc.), among others. The hydrophilic groups may be linked to the aromatic group directly or through any suitable linking moiety, which may be selected, for example, from alkyl groups (e.g., alkyl groups containing one carbon, two carbons, three carbons, four carbons, etc.), amide groups, ether groups, ester groups, urea groups, or urethane groups, among others.

In certain embodiments, the iodinated amino acids include amino acids that comprise one, two, three or more hydroxy-iodo-aromatic groups, such as hydroxy-iodo-phenyl groups or hydroxy-iodo-naphthyl groups. The hydroxy-iodo-aromatic groups may be selected, for example, from mono-hydroxy-mono-iodo-phenyl groups, mono-hydroxy-di-iodo-phenyl groups, mono-hydroxy-tri-iodo-phenyl groups, mono-hydroxy-tetra-iodo-phenyl groups, di-hydroxy-mono-iodo-phenyl groups, di-hydroxy-di-iodo-phenyl groups, di-hydroxy-tri-iodo-phenyl groups, tri-hydroxy-mono-iodo-phenyl groups, and tri-hydroxy-di-iodo-phenyl groups, among others. A few specific examples of iodine-containing amino acids for use in the present disclosure include the following: iodo-phenylalanine,

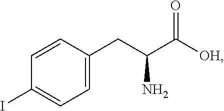 [SMILES: N[C@@H](Cc1ccc(I)cc1)C(=O)O]

which comprises a mono-iodo-phenyl group, monoiodotyrosine,

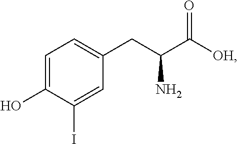 [SMILES: N[C@@H](Cc1ccc(O)c(I)c1)C(=O)O]

which comprises a mono-iodo-phenyl group, specifically, a mono-hydroxy-mono-iodo-phenyl group, diiodotyrosine,

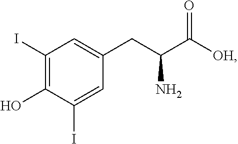 [SMILES: N[C@@H](Cc1cc(I)c(O)c(I)c1)C(=O)O]

which comprises a mono-hydroxy-di-iodo-phenyl group, diiodothyronine,

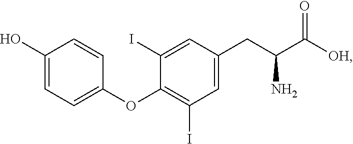 [SMILES: N[C@@H](Cc1cc(I)c(Oc2ccc(O)cc2)c(I)c1)C(=O)O]

which comprises a di-iodo-phenyl group and a hydroxy-phenyl group, triiodothyronine also known as T3, which

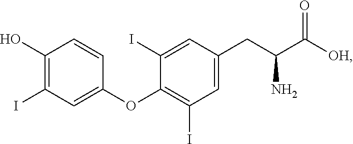 [SMILES: N[C@@H](Cc1cc(I)c(Oc2ccc(O)c(I)c2)c(I)c1)C(=O)O]

comprises a di-iodo-phenyl group and a mono-hydroxy-mono-iodo-phenyl group, tetraiodothyronine also known as thyroxine or T4,

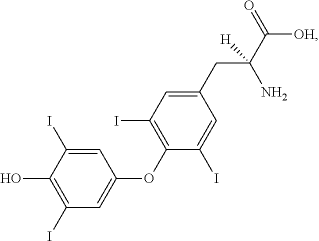 [SMILES: N[C@@H](Cc1cc(I)c(Oc2cc(I)c(O)c(I)c2)c(I)c1)C(=O)O]

which comprises a di-iodo-phenyl group and a mono-hydroxy-di-iodo-phenyl group, iodo-phenylalanine, and 6-iodo-L-DOPA, which comprises a di-hydroxy-mono-iodo-phenyl group, among others. Many of these iodinated amino acids are relatively water soluble, and some, including 3,5-diiodotyrosine and L-thyroxine, are well-studied as monomers for bioerodible polymers.

In various embodiment a hydroxy-terminated linear hydrophilic polymer or a hydroxy-terminated multi-arm hydrophilic polymer is reacted with a carboxylic acid group of an iodinated amino acid the presence of a suitable coupling agent (e.g., a carbodiimide coupling agent such as N,N′-dicyclohexylcarbodiimide (DCC), 1-ethyl-3-(3-dimethyl propyl) carbodiimide (EDC), 1,3-diisopropylcarbodiimide (DIC), N-hydroxybenzotriazole (HOBt), BOP reagent, and/or another coupling agent) to form an ester linkage. To prevent the amino group of the amino acid from reacting with the carboxylic acid group of the amino acid, the amino groups of the amino acid are protected using a suitable protective group. Examples of amine protective groups for use in conjunction with amide coupling reactions include tert-butoxycarbonyl (Boc) groups, carboxybenzyl (CBz) or (Z) groups, trifluoroacetyl (TFA) groups, and 9-fluorenylmethoxycarbonyl (Fmoc) groups, among others. Several commercially available Boc-protected iodinated amino acids are shown here:

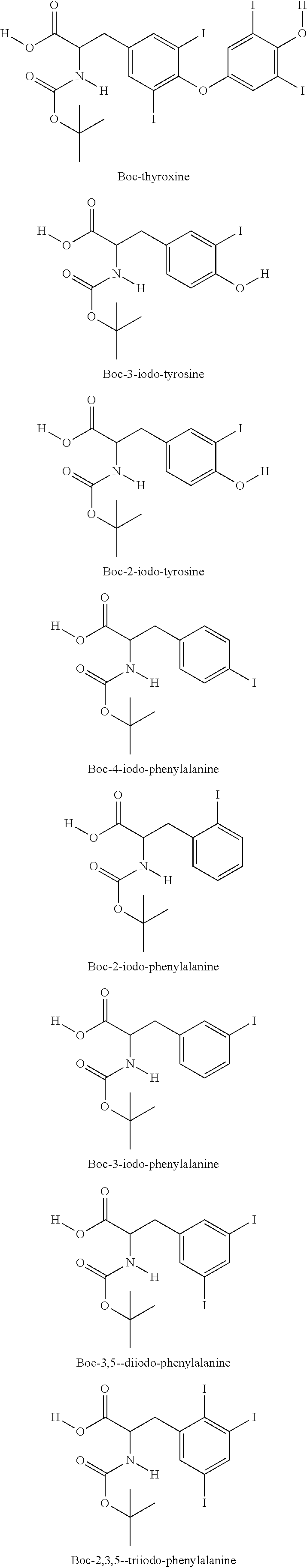

After amide coupling is complete, the resulting protected amino acid reside can be deprotected using known techniques, such as acid deprotection (e.g., HCl, trifluoroacetic acid, etc.) for Boc groups, hydrogenation (e.g., H₂/Pd-C) for CBz groups, base deprotection (e.g., with NaOH, KOH, etc.) for TFA groups, or amine deprotection (e.g., piperidine, piperazine, cyclohexylamine, ethanolamine, etc.) for Fmoc groups.

The deprotected amino group of the terminal amino-acid residue of the amino-acid-terminated linear hydrophilic polymer or the amino-acid-hydroxy-terminated multi-arm hydrophilic polymer is then available for further reaction. In various embodiments, the deprotected amino group is reacted in a ring-opening reaction with a cyclic anhydride to form a carboxylic acid group that is linked to the amino-acid residue through an amide-containing linkage. Several specific examples of anhydrides are listed in the following table.

| Species | CAS # |
|---|---|
| Succinic anhydride | 108-30-5 |
| Glutaric anhydride | 108-55-4 |
| Adipic anhydride | 2035-75-8 |
| Pimelic anhydride | 10521-07-0 |
| Malonic anhydride | 15159-48-5 |
| Itaconic anhydride | 2170-03-8 |
| Maleic anhydride | 108-31-6 |
| 3-Oxabicyclo[3.1.0]hexane-2,4-dione | 5617-74-3 |
| 3-Oxabicyclo[3.2.0]heptane-2,4-dione | 4462-96-8 |
| 3-Methylglutaric anhydride | 4166-53-4 |
| Methylsuccinic anhydride | 4100-80-5 |
| 2,2-Dimethylsuccinic anhydride | 17347-61-4 |
| 6,6-Dimethyl-3-oxabicyclo[3.1.0]hexane-2,4-dione | 67911-21-1 |
| Dihydro-4,4-dimethyl-2H-pyran-2,6(3H)-dione | 4160-82-1 |
| Diglycolic anhydride | 4480-83-5 |
| Oxane-2,4,6-trione | 10521-08-1 |
| Dihydro-4-(2-methylpropyl)-2H-pyran-2,6(3H)-dione | 185815-59-2 |

A reactive moiety, for example, an electrophilic moiety, may then be linked to the carboxylic acid group of the carboxylic-acid-substituted amino-acid residue arising from the ring-opening reaction.

In some embodiments, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic acid group of the carboxylic-acid-substituted amino-acid residue in the presence of a suitable coupling agent (e.g., a carbodiimide coupling agent such as N,N′-dicyclohexylcarbodiimide (DCC), 1-ethyl-3-(3-dimethyl'propyl) carbodiimide (EDC), N-hydroxybenzotriazole (HOBt), BOP reagent, and/or another coupling agent) to form a reactive cyclic imide ester (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to the amino-acid residue, more particularly, a reactive cyclic imide ester that is linked to the amino-acid residue through the amide-containing linkage that was formed in the ring-opening reaction described above. In this way, a number of activated ester groups can be formed.

For example, in the particular case of N-hydroxysuccinimide as an N-hydroxy cyclic imide compound, exemplary reactive moieties include those that comprise succinimidyl glutaramide groups, succinimidyl malonamide groups, succinimidyl succinamide groups, succinimidyl adipamide groups, succinimidyl pimelamide groups, and succinimidyl diglycolamide groups, among others. In the particular case of HONB as an N-hydroxy cyclic imide compound, exemplary reactive end groups include bicyclo[2.2.1]ept-5-ene-2,3-dicarboxylic acid imidyl malonamide groups, bicyclo[2.2.1]hept-5-ene-2,3-dicarboxylic acid imidyl glutaramide groups, bicyclo[2.2.1]hept-5-ene-2,3-dicarboxylic acid imidyl succinamide groups, bicyclo[2.2.1]hept-5-ene-2,3-dicarboxylic acid imidyl adipamide groups, bicyclo[2.2.1]hept-5-ene-2,3-dicarboxylic acid imidyl pimelamide groups, and bicyclo[2.2.1]hept-5-ene-2,3-dicarboxylic acid imidyl diglycolamide groups, among others. In the particular case of N-hydroxymaleimide as an N-hydroxy cyclic imide compound, exemplary reactive end groups include maleimidyl malonamide groups, malcimidyl glutaramide groups, maleimidyl succinamide groups, maleimidyl adipamide groups, maleimidyl pimelamide groups, and malcimidyl diglycolamide groups, among others. In the particular case of N-hydroxyglutarimide as an N-hydroxy cyclic imide compound, exemplary reactive end groups include glutarimidyl malonamide groups, glutarimidyl glutaramide groups, glutarimidyl succinamide groups, glutarimidyl adipamide groups, glutarimidyl pimelamide groups, glutarimidyl diglycolamide groups, among others. In the particular case of N-hydroxyphthalimide as an N-hydroxy cyclic imide compound, exemplary reactive end groups include phthalimidyl malonamide groups, phthalimidyl glutaramide groups, phthalimidyl succinamide groups, phthalimidyl adipamide groups, phthalimidyl pimelamide groups, and phthalimidyl diglycolamide groups, among others.

In some embodiments, the preceding reaction scheme can be used to form N-(cyclic-imidyl-oxycarbonyl-C₁-C₆-alkyl-carbonyl)-substituted iodinated amino acid groups, N-(cyclic-imidyl-oxycarbonyl-C₂-C₆-alkenyl-carbonyl)-substituted iodinated amino acid groups, and N-(cyclic-imidyl-oxycarbonyl-C₂-C₆-alkyloxyalkyl-carbonyl)-substituted iodinated amino acid groups, among others.

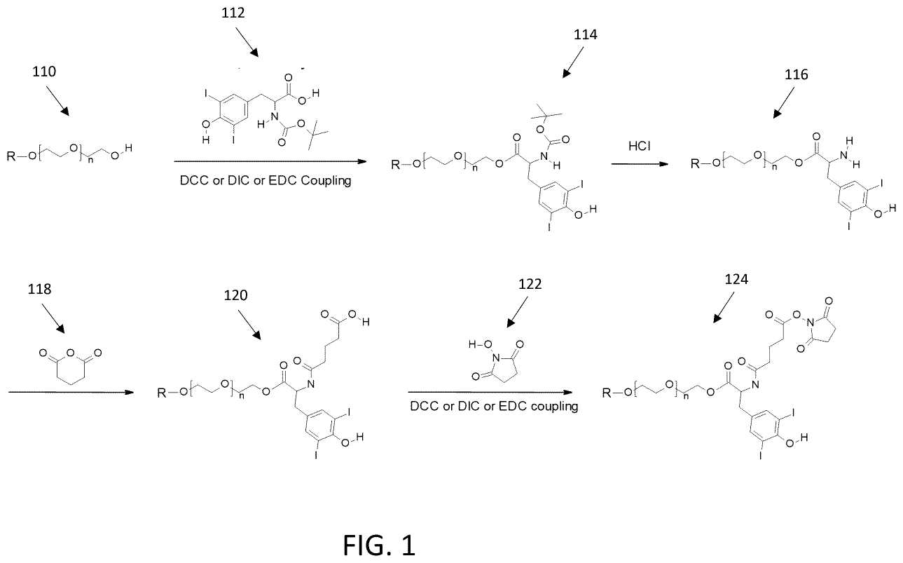

A particular embodiment will now be described with reference to FIG. 1, which employs a commercially available hydroxy-terminated multi-arm polymer, specifically, hydroxy-terminated eight-arm PEG 110, where R designates a core region and may correspond, for example, to a polyol residue such as a tripentaerythritol residue or a hexaglycerol residue among others, and n represents and integer and may have a value ranging from 5 to 500. It is noted that only a single arm is shown attached to the core, and the remaining arms are not illustrated. In a first step, the hydroxy-terminated eight-arm PEG 110 is reacted with an amine-protected iodinated amino acid, specifically, Boc-3,5-diiodotyrosine 112 (Boc-DIT, CAS#62129-53-7) in an ester coupling reaction to provide a multi-arm polymer that is terminated with amine-protected iodinated amino-acid residues, specifically, a Boc-protected diiodotyrosine-terminated 8-arm polymer, referred to herein as 8-arm-PEG-Boc-DIT 114. The ester coupling reaction may be conducted using a suitable coupling agent such as DCC, DIC, or EDC. The resulting ester group provides a hydrolysable linkage within the multi-arm polymer. Then, the protection group of the amine-protected iodinated amino-acid-residue-terminated multi-arm polymer is removed. Specifically, the Boc protection group of the 8-arm-PEG-Boc-DIT 114, is removed using a suitable acid such as HCl or trifluoroacetic acid to obtain a multi-arm polymer having arms that are terminated with an amino-containing iodinated amino-acid residue, specifically, a diiodotyrosine-terminated 8-arm polymer, referred to herein as 8-Arm-PEG-DIT 116. The amino groups of the iodinated amino-acid-terminated multi-arm polymer (i.c., the 8-Arm-PEG-DIT 116) are then reacted in a ring opening reaction with an acid anhydride, specifically, glutaric anhydride 118, to form a carboxylic-acid-substituted polymer in which carboxylic acid groups are linked to the iodinated amino-acid residues through an amide linkage, the particular multi-arm polymer of FIG. 1 being referred to hercin as 8-Arm-PEG-DIT-GA 120. Lastly, an N-hydroxy-cyclic-imide compound, specifically, N-hydroxysuccinimide 122, is coupled with the 8-Arm-PEG-DIT-GA 120 to produce a cyclic-imide-ester-substituted iodinated-amino-acid-residue-terminated multi-arm polymer, specifically, a succinimidyl-glutaramide-substituted 3,5-diiodotyrosine-residue-terminated 8-arm PEG, referred to herein as 8-Arm-PEG-DIT-SG 124. Stated another way, an N-(succinimidyl-oxycarbonyl-propyl-carbonyl)-3,5-diiodotyrosine-residue-terminated 8-arm PEG is produced.

The strategy shown in FIG. 1 is widely applicable to other hydroxy-terminated polymers, including linear PEG and hydroxy-terminated linear and multi-arm polymers having hydrophilic polymer segments besides PEG segments, such as those disclosed above.

In some aspects, the present disclosure provides radiopaque hydrogels that comprise a crosslinked reaction product of (a) a radiopaque, reactive polymer as described hereinabove (e.g., a radiopaque, reactive linear polymer and/or a radiopaque, reactive multi-arm polymer) and (b) a compound having a plurality of reactive nucleophilic moieties (e.g. amine moieties and/or thiol moieties, among others). Such hydrogels may be formed either in vivo or ex vivo.

In particular embodiments, the compound having a plurality of reactive nucleophilic moieties is a polyamino compound. In general, polyamino compounds suitable for use in the present disclosure include, for example, small molecule polyamines (e.g., containing at least two amine groups, for instance, from 3 to 20 amine groups or more in certain embodiments), polymers having amine side groups, and branched polymers having amine end groups, including dendritic polymers having amine end groups. Polyamino compounds suitable for use in the present disclosure include those that comprises a plurality of —(CH₂)ₓ—NH₂ groups where x is 0, 1, 2, 3, 4, 5 or 6. Polyamino compounds suitable for use in the present disclosure include polyamino compounds that comprise basic amino-acid residues, including residues of amino acids having two or more primary amine groups, such as lysine and ornithine, for example, polyamines that comprise from 2 to 10 lysine and/or ornithine amino-acid residues (e.g., dilysine, trilysine, tetralysine, pentalysine, diornithine, triornithine, tetraornithine, pentaornithine, etc.).

Particular examples of polyamino compounds which may be used as the

polyamino compound include ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, and poly allyl amine), among others among others.

In some embodiments, the polyamino compounds may be substituted with one or more radiopaque atoms such as iodine or bromine.

In various embodiments, the crosslinked reaction products of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values), for example, when measured on a bench-top micro CT system such as XtremeCT from Scanco Medical (Wangen-Brüttisellen, Switzerland) or similar.

Such crosslinked products may be formed in vivo (e.g., using a delivery device like that described below), or such crosslinked products may be formed ex vivo and subsequently administered to a subject. Such crosslinked products can be used in a wide variety of biomedical applications, including implants, medical devices, and pharmaceutical compositions.

In some aspects of the present disclosure, systems are provided that are configured to deliver (a) a polyamino compound (although a polyamino compound is described herein, it will be appreciated that other compounds having a plurality of reactive nucleophilic moieties may be employed) and (b) a radiopaque, reactive polymer as described herein (e.g., a reactive linear polymer and/or a radiopaque, reactive multi-arm polymer). The polyamino compound and the radiopaque, reactive polymer are combined under conditions such that the amino groups of the polyamino compound and the reactive moieties of the radiopaque, reactive polymer crosslink with one another. In certain embodiments, those conditions comprise an environment having a basic pH, for example, a pH ranging from about 8.5 to about 12. Such systems can be used to form radiopaque crosslinked hydrogels, either in vivo or ex vivo.

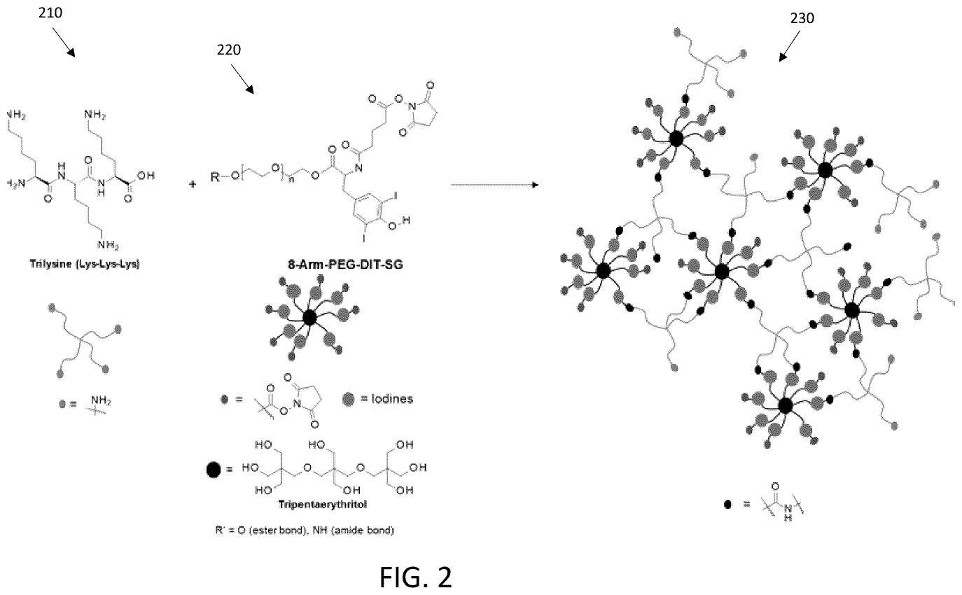

A particular example of a crosslinking reaction is shown in FIG. 2 which shows a succinimidyl-glutaramide-substituted 3,5-diiodotyrosine-residue-terminated 8-arm PEG (220) like that of FIG. 1 being covalent crosslinked with trilysine (210) under basic conditions to form a radiopaque hydrogel product (230).

In some aspects of the present disclosure, a system is provided that comprises (a) a first composition that comprises a polyamino compound, for example, as described herein (although a polyamino compound is described herein, it will be appreciated that other compounds having a plurality of reactive nucleophilic moieties may be employed) and (b) a second composition that comprises a radiopaque, reactive polymer (e.g., a reactive linear polymer and/or a radiopaque, reactive multi-arm polymer) as described herein,, wherein when the first and second compositions are combined, covalent crosslinks form between the reactive polymer and the polyamino compound, creating a hydrogel.

The first composition may be a first fluid composition comprising the polyamino compound or a first dry composition that comprises the polyamino compound, to which a suitable fluid such as water for injection, saline, etc. can be added to form a first fluid composition. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the radiopaque, reactive polymer or a second dry composition that comprises the radiopaque, reactive polymer, to which a suitable fluid such as water for injection, saline, etc. can be added to form a second fluid composition. In addition to the radiopaque, reactive polymer, the second composition may further comprise additional agents, including therapeutic agents, imaging agents, colorants, tonicity adjusting agents, suspension agents, wetting agents, and pH adjusting agents as described below.

In some embodiments, the polyamino compound is initially combined with the radiopaque, reactive polymer under conditions where crosslinking between the reactive moieties of the radiopaque, reactive polymer and the amino groups of the polyamino compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between same, thereby forming the crosslinked product.

In particular embodiments, the system comprises (a) a first composition that comprises a polyamino compound as described hereinabove, (b) a second composition that comprises a radiopaque, reactive polymer as described hereinabove, and (c) a third
composition, specifically, an accelerant composition, that contains an accelerant that is configured to accelerate a crosslinking reaction between the polyamino compound and the radiopaque, reactive polymer.

The first composition may be a first fluid composition comprising the polyamino compound that is buffered to an acidic pH or a first dry composition that comprises the polyamino compound and acidic buffering composition, to which a suitable fluid such as water for injection, saline, etc. can be added to form a first fluid composition comprising the polyamino compound that is buffered to an acidic pH. In some embodiments, for example, the acidic buffering composition may comprise monobasic sodium phosphate, among other possibilities. The first fluid composition comprising the polyamino compound may have a pH ranging, for example, from about 3 to about 6.5. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the radiopaque, reactive polymer or a second dry composition that comprises the radiopaque, reactive polymer from which a fluid composition is formed, for example, by the addition of a suitable fluid such as water for injection, saline, or the first fluid composition comprising the polyamino compound that is buffered to an acidic pH. In addition to the radiopaque, reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the polyamino compound that is buffered to an acidic pH and the second composition comprises a dry composition that comprises the radiopaque, reactive polymer. The first composition may then be mixed with the second composition to provide a prepared fluid composition that is buffered to an acidic pH and comprises the polyamino compound and the radiopaque, reactive polymer. In a particular example, a syringe may be provided that contains the first fluid composition comprising the polyamino compound that is buffered to an acidic pH, and a vial may be provided that comprises the dry composition (e.g., a powder) that comprises the radiopaque, reactive polymer. The syringe may then be used to inject the first fluid composition into the vial containing the radiopaque, reactive polymer to form a prepared fluid composition that contains the polyamino compound and the radiopaque, reactive polymer, which can be withdrawn back into the syringe for administration.

The third composition may be a fluid accelerant composition that is buffered to a basic pH or a dry composition that comprises a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the polyamino compound and the radiopaque, reactive polymer as described above (as well as additional agents in some cases), and a fluid accelerant composition that is buffered to basic pH as described above (which may include additional agents in some cases), may be combined form crosslinked hydrogels, either in vivo or ex vivo.

In some particular embodiments of the present disclosure, a kit is provided that include a first reservoir (e.g., a vial or syringe barrel) containing a first composition comprising a polyamino compound as described herein and an acidic buffer, a second reservoir (e.g., a vial or syringe barrel) containing a second composition comprising containing a radiopaque, reactive polymer as described herein, a third reservoir (e.g., a vial or syringe barrel) containing a third composition comprising a buffered accelerant as described herein, additional apparatus, as required, for combining the first and second compositions to provide a prepared fluid composition that is buffered to an acidic pH and comprises the polyamino compound and the radiopaque, reactive polymer, and additional apparatus for combining and delivering the prepared fluid composition and buffered accelerant to a patient.

In more particular embodiments, the kit may comprise a vial containing a radiopaque, reactive polymer as described herein in dry (e.g., powdered) form, a first syringe containing a fluid composition comprising a polyamino compound as described herein that is buffered to an acidic pH, a second syringe containing a buffered accelerant solution as described herein, a needle and/or tube, a Y-connector, a syringe holder, a plunger cap and a vial adapter. Such components may be placed in sterile packaging, for example, in one or more packaged sterile trays.

The compositions described herein may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO₂, gamma, x-ray or electron beam irradiation.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.c., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents (beyond the radiopaque iodine atoms that are present) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

A prepared fluid composition that is buffered to an acidic pH and comprises the polyamino compound and the radiopaque, reactive polymer as described above, and a fluid accelerant composition that is buffered to basic pH as described above, may be combined form radiopaque crosslinked hydrogels, either in vivo or ex vivo.

In various embodiments, a system is provided that includes one or more delivery devices for delivering first and second compositions to a subject.

In some embodiments, the system may include a delivery device that comprises a first reservoir that contains a first composition that comprises a polyamino compound as described above and a second reservoir that contains a second composition that comprises a radiopaque, reactive polymer that comprises a plurality of reactive moieties that are reactive with the amino moieties of the polyamino compound as described above.

In some embodiments, the system may include a delivery device that comprises a first reservoir that contains a first composition that comprises the polyamino compound and the radiopaque, reactive polymer and is buffered to an acidic pH, such as the prepared fluid composition previously described, and a second reservoir that contains second composition, such as the fluid accelerant composition previously described.

In either case, during operation, the first composition and second composition are dispensed from the first and second reservoirs and combined, whereupon the polyamino compound and the radiopaque, reactive polymer and crosslink with one another to form a radiopaque crosslinked hydrogel.

In either case, during operation, the first fluid composition and second fluid composition are dispensed from the first and second reservoirs and combined, whereupon the polyamino compound and the radiopaque, reactive polymer crosslink with one another to form a radiopaque crosslinked hydrogel.

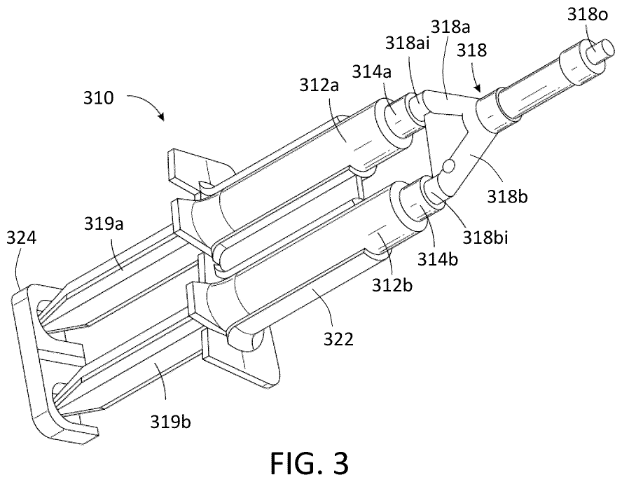

In particular embodiments, and with reference to FIG. 3, the system may include a delivery device 310 that comprises a double-barrel syringe, which includes a first barrel 312a having a first barrel outlet 314a, which first barrel contains a first fluid composition as described above, a first plunger 319a that is movable in the first barrel 312a, a second barrel 312b having a second barrel outlet 314b, which second barrel 312b contains a second fluid composition as described above, and a second plunger 319b that is movable in the second barrel 312b. In some embodiments, the device 310 may further comprise a mixing section 318 (e.g., a Y-connector) having a first mixing section inlet 318ai in fluid communication with the first barrel outlet 314a, a second mixing section inlet 318bi in fluid communication with the second barrel outlet 314b, and a mixing section outlet 318o. Also shown are a syringe holder 322 configured to hold the first and second syringe barrels 312a, 312b, in a fixed relationship and a plunger cap 324 configured to hold the first and second plungers 319a, 319b in a fixed relationship.

In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a radiopaque crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (c.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a radiopaque crosslinked hydrogel is ultimately formed at the administration location.

During and/or after administration, the compositions of the present disclosure can be imaged using a suitable imaging technique. Typically, the imaging technique is an x-ray-based imaging technique, such as computerized tomography or X-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, radiopaque crosslinked hydrogels may be in any desired form, including a slab, a cylinder, a coating, or a particle. In some embodiments, the radiopaque crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Radiopaque crosslinked hydrogel particles formed using the above and other techniques may vary widely in size, for example, having an average size ranging from 50 to 950 microns.

In addition to a radiopaque crosslinked hydrogel as described above, radiopaque crosslinked hydrogel compositions in accordance with the present disclosure may contain additional agents, including therapeutic agents, imaging agents, colorants, tonicity adjusting agents, suspension agents, wetting agents, and pH adjusting agents as described above.

The crosslinked hydrogel compositions of the present disclosure may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO₂, gamma, x-ray or electron beam irradiation.

In various embodiments, kits are provided that include one or more delivery devices for delivering the radiopaque crosslinked hydrogel to a subject. Such systems may include one or more of the following: a syringe barrel, which may or may not contain a radiopaque crosslinked hydrogel as described herein; a vial, which may or may not contain a radiopaque crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the radiopaque crosslinked hydrogel may be provided in dry form (e.g., powder form) or in a form that is ready for injection, such as an injectable hydrogel form (e.g., a suspension of radiopaque crosslinked hydrogel particles).

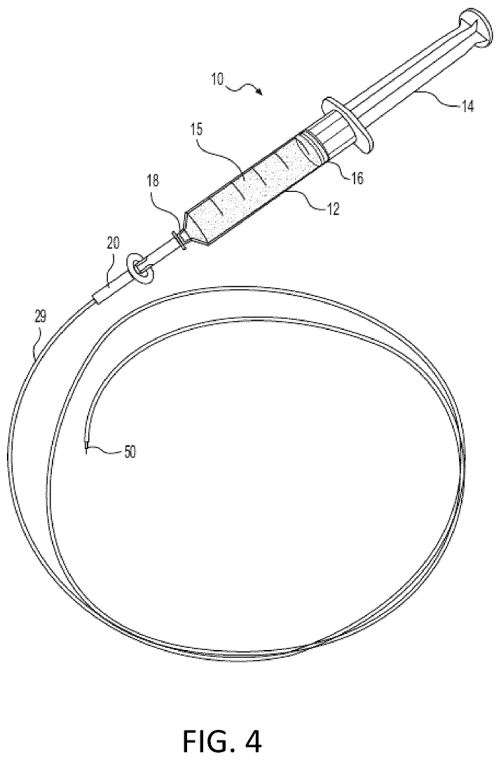

FIG. 4 illustrates a syringe 10 providing a reservoir for a radiopaque crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a radiopaque crosslinked hydrogel composition 15 for injection through the needle 50.

The radiopaque crosslinked hydrogel compositions described herein can be used for a number of purposes.

For example, radiopaque crosslinked hydrogel compositions can be injected to provide spacing between tissues, radiopaque crosslinked hydrogel compositions can be injected (e.g., in the form of blebs) to provide fiducial markers, radiopaque crosslinked hydrogel compositions can be injected for tissue augmentation or regeneration, radiopaque crosslinked hydrogel compositions can be injected as a filler or replacement for soft tissue, radiopaque crosslinked hydrogel compositions can be injected to provide mechanical support for compromised tissue, radiopaque crosslinked hydrogel compositions be injected as a scaffold, and/or radiopaque crosslinked hydrogel compositions can be injected as a carrier of therapeutic agents in the treatment of diseases and cancers and the repair and regeneration of tissue, among other uses.

During and/or after administration, the radiopaque crosslinked hydrogel compositions of the present disclosure can be imaged using a suitable imaging technique.

As seen from the above, the radiopaque crosslinked hydrogel compositions of the present disclosure may be used in a variety of medical procedures, including the following, among others: a procedure to implant a fiducial marker comprising a radiopaque crosslinked hydrogel, a procedure to implant a tissue regeneration scaffold comprising a radiopaque crosslinked hydrogel, a procedure to implant a tissue support comprising a radiopaque crosslinked hydrogel, a procedure to implant a tissue bulking agent comprising a radiopaque crosslinked hydrogel, a procedure to implant a therapeutic-agent-containing depot comprising a radiopaque crosslinked hydrogel, a tissue augmentation procedure comprising implanting a radiopaque crosslinked hydrogel, a procedure to introduce a radiopaque crosslinked hydrogel between a first tissue and a second tissue to space the first tissue from the second tissue.

The radiopaque crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Radiopaque crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A radiopaque, reactive polymer comprising (a) one or more hydrophilic polymer regions, the one or more hydrophilic polymer regions having first and second ends and comprising at least one hydrophilic polymer segment, (b) a plurality of iodinated amino-acid residues, wherein at least one of the plurality of iodinated amino-acid residues is linked to each of the hydrophilic polymer regions, and (c) a plurality of reactive moieties, wherein at least one of the plurality of reactive moieties is linked to each of the plurality of iodinated amino-acid residues and wherein the reactive moieties each comprise an electrophilic group.

2. The radiopaque, reactive polymer of claim 1, wherein the reactive polymer is a linear polymer, wherein the one or more hydrophilic polymer regions correspond to a central linear hydrophilic polymer region, wherein one of the plurality of iodinated amino-acid residues is linked to the first end of the central linear hydrophilic polymer region, and wherein one of the plurality of iodinated amino-acid residues is linked to the second end of the central linear hydrophilic polymer region.

3. The radiopaque, reactive polymer of claim 1, wherein the reactive polymer is a multi-arm polymer comprising a core region and a plurality of polymer arms, wherein the plurality of polymer arms each comprise one of the one or more hydrophilic polymer regions, and wherein the first end of each of the one of the one or more hydrophilic polymer regions is linked to the core region, and wherein the second end of each of the one of the one or more hydrophilic polymer regions is linked to one of the plurality of iodinated amino-acid residues.

4. The radiopaque, reactive polymer of claim 1, wherein each of the plurality of iodinated amino-acid residues is linked to one of the one or more hydrophilic polymer regions through an ester group and wherein each of the plurality of reactive moieties is linked to one of the plurality of iodinated amino-acid residues though an amide group.

5. The radiopaque, reactive polymer of claim 1, wherein each of the plurality of iodinated amino-acid residues comprises an iodinated aromatic group.

6. The radiopaque, reactive polymer of claim 5, wherein the iodinated aromatic group is a hydroxy-iodo-aromatic group.

7. The radiopaque, reactive polymer of claim 6, wherein the hydroxy-iodo-phenyl group is selected from a mono-hydroxy-mono-iodo-phenyl group, a mono-hydroxy-di-iodo-phenyl group, a mono-hydroxy-tri-iodo-phenyl group, a mono-hydroxy-tetra-iodo-phenyl group, a di-hydroxy-mono-iodo-phenyl group, a di-hydroxy-di-iodo-phenyl group, a di-hydroxy-tri-iodo-phenyl group, a di-hydroxy-tetra-iodo-phenyl group, a tri-hydroxy-mono-iodo-phenyl group, a tri-hydroxy-di-iodo-phenyl group, tri-hydroxy-tri-iodo-phenyl group, and a tri-hydroxy-tetra-iodo-phenyl group.

8. The radiopaque, reactive polymer of claim 1, wherein the iodinated amino-acid residues are selected from mono-iodophenylalanine, di-iodophenylalanine, tri-iodophenylalanine, mono-iodoiodotyrosine, di-iodoiodotyrosine, tri-iodoiodotyrosine, mono-iodothyronine, di-iodothyronine, tri-iodothyronine, and tetra-iodothyronine residues.

9. The radiopaque, reactive polymer of claim 1, wherein the electrophilic group is a cyclic imide ester group.

10. The radiopaque, reactive polymer of claim 9, wherein the cyclic imide ester group is selected from a succinimide ester group, a maleimide ester group, a glutarimide ester group, a diglycolimide ester groups, a phthalimide ester group, and a bicyclo[2.2.1]hept-5-ene-2,3-dicarboxylic acid imide ester group.

11. The radiopaque, reactive polymer of claim 1, wherein the radiopaque, reactive polymer comprises N-(cyclic-imidyl-oxycarbonyl-C₁-C₆-alkyl-carbonyl)-substituted iodinated amino acid residues, N-(cyclic-imidyl-oxycarbonyl-C₂-C₆-alkenyl-carbonyl)-substituted iodinated amino acid residues, or N-(cyclic-imidyl-oxycarbonyl-C₂-C₆-alkyloxyalkyl-carbonyl)-substituted iodinated amino acid residues.

12. The radiopaque, reactive polymer of claim 1, wherein the radiopaque, reactive polymer comprises succinimidyl glutaramide groups, succinimidyl malonamide groups, succinimidyl succinamide groups, succinimidyl adipamide groups, succinimidyl pimelamide groups, or succinimidyl diglycolamide groups.

13. The radiopaque, reactive polymer of claim 1, wherein the at least one hydrophilic polymer segment comprises one or more monomer residues selected from ethylene oxide, propylene oxide, N-vinyl pyrrolidone and oxazoline monomer residues.

14. The radiopaque, reactive polymer of claim 1, wherein the at least one hydrophilic polymer segment contains between 40 and 4000 monomer residues.

15. A system for forming a hydrogel composition that comprises (1) a compound having a plurality of nucleophilic moieties; and (2) a radiopaque, reactive polymer comprising (a) one or more hydrophilic polymer regions, the one or more hydrophilic polymer regions having first and second ends and comprising at least one hydrophilic polymer segment, (b) a plurality of iodinated amino-acid residues, wherein at least one of the plurality of iodinated amino-acid residues is linked to each of the hydrophilic polymer regions, and (c) a plurality of reactive moieties, wherein at least one of the plurality of reactive moieties is linked to each of the plurality of iodinated amino-acid residues and wherein the reactive moieties each comprise an electrophilic group.

16. The system of claim 15, wherein the compound having a plurality of nucleophilic moieties is a polyamino compound.

17. The system of claim 15, wherein the system comprises a first composition that comprises the compound having a plurality of nucleophilic moieties and a second composition that comprises the radiopaque, reactive polymer.

18. The system of claim 17, further comprising an accelerant composition.

19. The system of claim 15, further comprising a delivery device.

20. A method of treatment comprising administering to a subject a mixture that comprises (1) a radiopaque, reactive polymer comprising (a) one or more hydrophilic polymer regions, the one or more hydrophilic polymer regions having first and second ends and comprising at least one hydrophilic polymer segment, (b) a plurality of iodinated amino-acid residues, wherein at least one of the plurality of iodinated amino-acid residues is linked to each of the hydrophilic polymer regions, and (c) a plurality of reactive moieties, wherein at least one of the plurality of reactive moieties is linked to each of the plurality of iodinated amino-acid residues and wherein the reactive moieties each comprise an electrophilic group; and (2) a compound having a plurality of nucleophilic moieties, wherein the mixture is administered under conditions such that the compound having a plurality of nucleophilic moieties and the radiopaque, reactive polymer crosslink to form a hydrogel after administration.
